(19) United States
(12) Patent Application Publication
(10) Pub. No.: US 2005/0283044 A1
(43) Pub. Date: Dec. 22, 2005

(54) FLEXIBLE RING FOR PENIS

(76) Inventor: Wen-Liang Chang, Feng Shan (TW)

Correspondence Address: NIKOLAI & MERSEREAU, P.A., 900 SECOND AVENUE SOUTH, SUITE 820, MINNEAPOLIS, MN, 55402, US

(21) Appl. No.: 10/907,373

(22) Filed: Mar. 30, 2005

(30) Foreign Application Priority Data
Jun. 21, 2004 (CN) 200420066478.6

(51) Int. Cl. A61F 5/00

(52) U.S. Cl. 600/38

(57) ABSTRACT

The present invention is to provide a flexible ring comprising a cylindrical receptacle formed on an outer edge; a battery operated vibrator mounted in said receptacle; an annular flange around its inner edge; two opposite holes each through front and rear surfaces; wherein the ring is adapted to put on the base of an erect penis, the flange is adapted to tightly clamp the penis, and the holes are elastically deforming for increasing the resilience and the clamping force such that activating the vibrator will prolong the climax of sexual excitement by delaying ejaculation by the strong clamping force.

### BACKGROUND OF THE INVENTION

1. Field of Invention

The present invention relates to sex products and more particularly to a flexible ring adapted to tightly put on the base of an erect penis such that a subsequent sexual intercourse can be enjoyed satisfactorily by both parties because the ring can prolong the climax of sexual excitement by delaying ejaculation by the strong clamping force.

2. Related Art

Various sex products are available in the market. Some of them are beneficial, however, much of them are merely a gimmick of one sort or another. Another problem often bothers male is impotence. Drugs (e.g., Viagra) for improving or even eliminating impotence are also widely available throughout the world. However, side effect of taking such drug cannot be ignored since it is sometimes very serious. Hence, a need has arisen for a simple, cost effective device for improving impotence without the risk of taking medicine.

### SUMMARY OF THE INVENTION

It is therefore an object of the present invention to provide a flexible ring comprising a cylindrical receptacle formed on an outer edge; a battery operated vibrator mounted in said receptacle; an annular flange around its inner edge; two opposite holes each through front and rear surfaces; wherein the ring is adapted to put on the base of an erect penis, the flange is adapted to tightly clamp the penis, and the holes are elastically deforming for increasing the resilience and the clamping force such that activating the vibrator will prolong the climax of sexual excitement by delaying ejaculation by the strong clamping force.

The above and other objects, features and advantages of the present invention will become apparent from the following detailed description taken with the accompanying drawings.

### BRIEF DESCRIPTION OF THE DRAWINGS

FIG. 1 is a perspective view of a flexible ring in accordance with a preferred embodiment of the invention;

FIG. 2 is a sectional view taken along line 1-1 of FIG. 1; and

FIG. 3 is an environmental view of the ring in its section tightly put on the base of an erect penis prior to sexual intercourse.

### DETAILED DESCRIPTION OF THE INVENTION

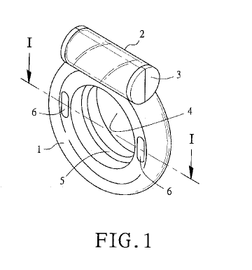

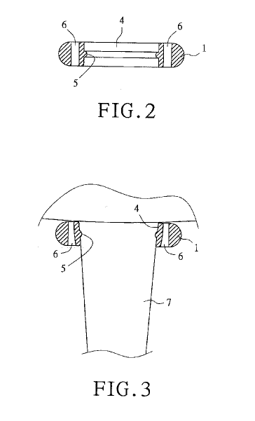

Referring to FIGS. 1 and 2, there is shown a ring 1 constructed in accordance with a preferred embodiment of the invention. The ring 1 is made of a suitable elastomeric material (i.e., flexible) and comprises an annular flange 5 formed around its inner edge, two opposite holes 6 each through front and rear surfaces, a cylindrical receptacle 2 formed on an outer edge, and a battery operated motor unit 3 mounted in the receptacle 2 such that the receptacle 2 can be served as a vibrator during use.

Referring to FIG. 3, use of the ring 1 of the invention will be described in detailed below. First, put the ring 1 on the base of an erect penis 7 by causing the penis 7 to pass through a central channel 4 of the ring 1. The penis 7 is tightly clamped by the flange 5 and the cross sectional area of the ring 1 is decreased according to the holes 6, thereby the holes are elastically deforming for increasing the resilience and the clamping force. In a subsequent sexual intercourse the ring 1 in cooperation with the activating motor unit 3 thus can prolong the climax of sexual excitement by delaying ejaculation by the strong clamping force. As an end, both parties can satisfactorily enjoy the sex.

While the invention herein disclosed has been described by means of specific embodiments, numerous modifications and variations could be made thereto by those skilled in the art without departing from the scope and spirit of the invention set forth in the claims.

1. A flexible ring comprising:
a cylindrical receptacle formed on an outer edge;
a battery operated vibrator mounted in said receptacle; and
wherein an annular flange being formed around an inner edge of the ring, thereby putting the ring on the base of an erect penis, the flange tightly clamps the penis such that activating the vibrator will prolong the climax of sexual excitement by delaying ejaculation by the clamping force.

2. A flexible ring comprising:
a cylindrical receptacle formed on an outer edge;
a battery operated vibrator mounted in said receptacle; and
wherein two opposite holes penetrating through front and rear surfaces of the ring to decrease the cross sectional area, thereby the holes are elastically deforming for increasing the resilience and the clamping force so as to improve the comfortableness for wearing.
